(19) United States
(12) Patent Application Publication
(10) Pub. No.: US 2007/0032648 A1
(43) Pub. Date: Feb. 8, 2007

(54) Triazines for improved lubricity and mineral dispersion

(76) Inventor: Thomas Daly, Chicago, IL (US)

Correspondence Address: Thomas Daly, House C, 1400 N. North Park Ave, Chicago, IL, 60610, US

(21) Appl. No.: 11/498,029

(22) Filed: Aug. 2, 2006

Related U.S. Application Data
(60) Provisional application No. 60/705,344, filed on Aug. 4, 2005.
(60) Provisional application No. 60/716,006, filed on Sep. 9, 2005.

(51) Int. Cl. C07D 251/02

(52) U.S. Cl. 544/180

(57) ABSTRACT

An oil soluble triazine that effectively disperses pigments and minerals into a hydrophobic solvent system. The triazine is the formaldehyde condensate of amines that are either alkyl, allyl or alkynal, that is either branched or linear that is from 2 to 22 carbons in length.

This application is related to, and claims priority from, U.S. Provisional application 60/705,344 filed Aug. 5, 2005 and hereby incorporates that application by reference. This application is also related to, and claims priority from, U.S. Provisional application 60/716,006 filed Sep. 9, 2005 and hereby incorporates that application by reference.

### BACKGROUND

1. Field of the Invention

The present invention relates to the field of triazines and more particularly to higher molecular weight triazines applied as lubricity aids and dispersants.

2. Description of the Problem Solved by the Invention

Often times minerals or other insoluble solids need to be dispersed into hydrophobic media, such as pigments into solvent-borne coatings systems. This is often accomplished by the addition of surface active material which are often comprised of volatile organic compounds, (VOCs), which are highly regulated due to their adverse environmental effects. The surface active compounds also often effect the coatings in adverse way by damaging the adhesion strength, or simply damaging the polymer film through the inclusion of surfactants that act like soap to dissolve the coating when severely stressed. The present invention teaches the use of a class of triazines that provide superior pigment dispersion and without damaging the film properties.

The present invention also finds utility in metal working fluids (MWFs) as a multifunctional additive. The present invention enhances the lubricity of typical hydrocarbon or ester metal working fluid bases. In addition, the present invention also sequesters H₂S that can form in an idle sump, typically over a weekend or other period were the sump may not be recirculated. This condition is commonly called, “Monday morning odor.” The use of the present invention will sequester the H2S and greatly reduce worker exposure to this highly toxic gas. The present invention also acts to inhibit the growth of the sulfate reducing bacteria that break down the sulfur compounds in the MWF or MWF components to H₂S.

### SUMMARY OF THE INVENTION

The present invention relates to an oil soluble triazine that can be utilized to disperse pigments, minerals, or other insolubles into a hydrophilic medium. Particular examples include, but are not limited to, pigments, lime dispersions for building materials, silica and other mineral dispersion for a wide variety of applications, including, but limited to, chemical, mechanical planerization in the manufacture of silica based wafers, ceramics or metals.

When the present invention is applied in coatings, the use of unsaturated amines yields a dispersant that can be cross-linked into reactive resin systems such as alkyds and acrylics. This greatly enhances the durability of the coating by eliminating the need for a surfactant that will stay in the film and act as a soap and disrupt the polymeric film under extreme conditions. This also leads to improved adhesion as surfactants do not remain to interfere at the surface with the resin/surface interface. Additionally, the present invention does not contribute to VOC and yields a zero VOC dispersion if an exempt solvent, or no solvent is used.

The present invention also finds use as a lubricity additive in MWFs, and the control and sequestering of hydrogen sulfide in metal working fluids, or any hydrocarbon, hydrocarbon based system, emulsion, or aqueous system where hydrogen sulfide control would be desirable, in use, transport or storage. The triazine is, in general, but not limited to, a six membered ring structure with three nitrogen atoms and three carbon atoms, with the nitrogen and carbon atoms alternating positions around the ring, the nitrogens shall have an alkyl, allyl or alkynal tail that is either branched or linear that is from 3 to 22 carbons in length. Each nitrogen may have the same or different hydrocarbon tail. Such product is the condensation product of formaldehyde and a primary amine whose structure is shown below: 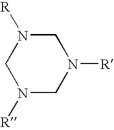
where R, R′ and R″ may be the same or different and are either alkyl, allyl or alkynal, that is either branched or linear that is from 2 to 22 carbons in length.

The invention also includes the condensation product of primary ether amines that are alkyl, allyl, or alkynal, that are branched or linear and are from 2 to 22 carbons in length.

While a less effective solution, it is also within the scope of the invention to use amines, polyamines, ether amines, or ether polyamines that are linear or branched, alkyl, allyl, or alkynal with from 2 to 22 carbons, alone, as mixtures, as partially or fully neutralized salts, or in combinations or in combination with the class of triazines above to sequester hydrogen sulfide in hydrocarbon based materials in transit or storage. The use of amines and ether amines with longer chain lengths are generally preferred, both as sequesterants in there in own right, as well as when used as starting materials for the triazines, due to their reduced volatility and increased partitioning into the hydrocarbon phase. If desired, shorter carbon chained amines can be used to increase the partitioning into the aqueous phase, or in the case of the longer chain amines and ether amines, the amine can be salted, partially or fully to obtain the partitioning coefficient desired.

Triazines are widely known to act as antimicrobials, and the oil soluble triazines contained in the present invention exhibit antimicrobial properties, further reducing the production of sulfate reducing bacteria present in the rag layer, thus improving the efficacy of the sequesterant. The triazines contained in the invention also are expected to show efficacy against a type of bacteria called mycobacteria. The >70 species of mycobacteria include Mycobacterium tuberculosis, which is responsible for tuberculosis and Mycobacterium leprae, which is responsible for leprosy. Other mycobacteria are responsible for opportunistic infections, especially in people with AIDS. It is believed that the present invention has great promise as the basis of a therapy to treat these diseases, either alone, or in combination with established therapies.

### DESCRIPTION OF THE FIGURES

FIG. 1 shows the synthesis of tallow triazine.

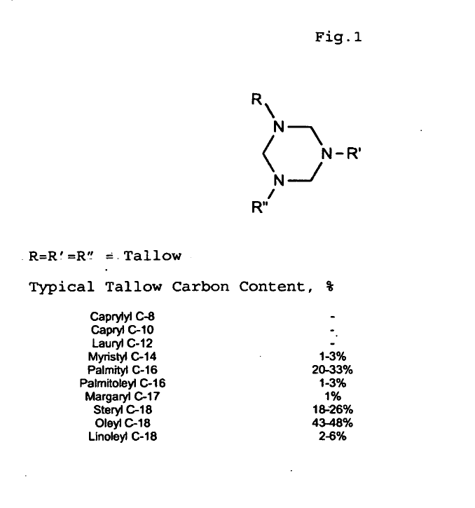

FIG. 2 shows the synthesis of an ether amine triazine.

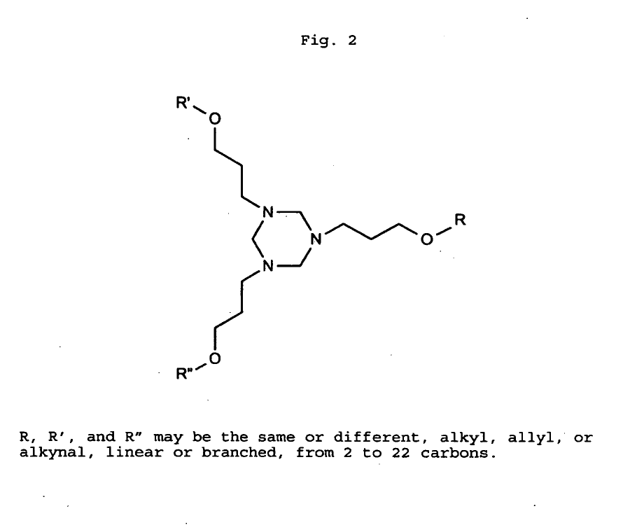

### DETAILED DESCRIPTION OF THE INVENTION

The preferred embodiment of the invention is the condensation product of primary tallow amine and paraformaldehyde added in a sufficient quantity to improve lubricity to the extent desired. When used primarily to sequester hydrogen sulfide and prevent the release of toxic fumes from the storage vessel or transportation vessel, a lesser amount is expected to be needed in all cases but the highest sulfur containing MWF. Further, used in combinations with water soluble triazines, such as the formaldehyde condensation product of monoethanol amine and formaldehyde, to sequester hydrogen sulfide and to control microbial activity which in turn reduces or eliminates the ongoing production of hydrogen sulfide by sulfate reducing bacteria creates a more effective solution and the combination is within the scope of this invention as is the combination with any antimicrobial. Additionally, combinations with oil soluble amines, polyamines, ether amines, and/or ether polyamines can improve the economics and is within the scope of the invention as is the use of branched or linear allyl, alkyl, or alkynal amines or ether amines, alone or in combination.

### EXAMPLE 1

### Production of a Triazine Lubricity Aid for MWFs

To a vessel equipped with agitation, heating, vacuum, and condensation capabilities was added 346 g of primary tallow amine (1.30 moles) and 36.2 g of paraformaldehyde, 92% (1.30 moles). The vessel was evacuated and agitation started, followed by heating to 80 C. The vessel was held at 80 C for two hours, by which time 24 g of water had been recovered and at which time no further water was being generated. 354 g of a homogenous, low viscosity, liquid was recovered.

I claim:

1) A pigment dispersant of the following formula: 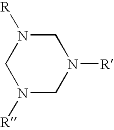
where R, R′ and R″ may be the same or different and are either alkyl, allyl or alkynal, that is either branched or linear that is from 2 to 22 carbons in length.
